(19) United States
(12) Patent Application Publication
(10) Pub. No.: US 2023/0071069 A1
(43) Pub. Date: Mar. 9, 2023

(54) Electric and Magnetic Neuromodulation

(71) Applicants: Derek H. Jin, Yorba Linda, CA (US); Conway Ho, La Palma, CA (US)

(72) Inventors: Derek H. Jin, Yorba Linda, CA (US); Conway Ho, La Palma, CA (US)

(21) Appl. No.: 17/896,072

(22) Filed: Aug. 26, 2022

Related U.S. Application Data
(60) Provisional application No. 63/238,218, filed on Aug. 29, 2021.

(51) Int. Cl. A61N 2/02; A61N 2/00

(52) U.S. Cl. CPC A61N 2/02; A61N 2/006; A61N 2/008; A61N 2/002

(57) ABSTRACT

Neuromodulation is achieved by administering variable pulse electric or magnetic stimulation to a patient in need thereof. The neuromodulation can inhibit or enhance neuronal-synaptic transmission or muscle-synaptic transmission between neuron and muscle fibers The variable pulse electric or magnetic stimulation varies based on one or more mean and standard deviations of biological variables. Biological variables include heart rate variability, EEG variability, EMG variability and the frequency of activity measured in the spinal cord. The invention also includes devices that deliver the variable electric or magnetic pulses to the patient. Magnetic stimulation can be provided by electromagnetic or solid magnet stimulation.

The presentation application claims benefit to Provisional application Ser. 63/238,218 filed Aug. 29, 1921 which is incorporated herein by reference

The present invention relates to non-invasive neuromodulation using electric and magnetic stimulation. The electric and magnetic stimulation can inhibit neuronal-synaptic transmission or neuronal-muscular junction transmission. The electric and magnetic stimulation can also enhance neuronal synaptic and neuronal-muscular junction transmission Additionally, the present invention is directed to devices that deliver variable electric and magnetic pulses of the present invention.

### BACKGROUND OF THE INVENTION

Neuromodulation is a medical procedure that acts directly upon nerves to either enhance or inhibit nerve activity. Historically, neuromodulation was accomplished by delivering electrical stimulation or pharmaceutical agents in small quantities directly to a target area in order to affect the activities in secondary region(s) associating with the target area. Neuromodulation can be used in most every area of the body and treats a variety of diseases and symptoms including, but not limited to, nerve and muscle pain both acute and chronic, headaches, tremors, Parkinson's Disease, spinal cord damage, migraine, epilepsy, and urinary incontinence. Current magnetic therapies employ a single frequency stimulation.

### SUMMARY OF THE INVENTION

Briefly, in accordance with the present invention, neuromodulation is achieved in a patient by administering electric or magnetic stimulation to the patient using variable magnetic or electric pulses. Magnetic stimulation includes both electromagnetic and solid magnet stimulation. Preferably, the magnetic pulses are random in the forms of noise pattern, such as for example, white noise, pink noise, violet noise, blue noise, brown noise and red noise. The present treatments can inhibit or enhance the neuronal-synaptic transmission or neuronal-muscular junction (NMJ) transmission. The present invention can be used to treat a variety of indications including but not limited to pain or a variety of psychological disorders and conditions including but not limited to, PTSD, stroke, Alzheimer's Disease, autism, addiction, depression, sleep disorders, performance deficiencies and the like.

In one embodiment the treatment protocol involves first measuring a biometric character of a patient resulting in a biometric data set, comparing the biometric data set with a normative database to determine if the patient needs neuromodulation, analyzing the biometric data set to identify characteristics of distribution probabilities of one or more variables resulting in mean and standard deviation of pulse period values, and then administering magnetic stimulation to the patient wherein the magnetic stimulation comprises variable pulses based on one or more mean and standard deviations of the biological variables. The biological variables include, but are not limited to, heart rate variability, EEG variability, EMG variability and the frequency of activity variability measured in the spinal cord. The neuromodulation can inhibit or enhance nerve transmission between neurons or neuron muscle junctions depending on the variable electromagnetic pulses employed.

Of particular interest in practicing the present invention, both acute and chronic pain are controlled by administering a high frequency band-limited random electromagnetic pulse stimulation in a preferred noise pattern. Additionally, a personalized transcranial magnetic stimulation controlled by trains of random TTL pulses normalized by Gaussian Distribution with an individual's EEG mean period and its standard deviations are administered to a patient to treat psychiatric disorders or other neurological conditions.

Medical conditions that can be treated according to the present invention include but are not limited to muscle twitching, cramps, aches and pains, neck, shoulder, and arm pain caused by rheumatoid arthritis, osteoporosis, fibromyalgia, spondylosis, herniated cervical disk, and spinal stenosis; back pain caused by muscle or ligament strain, bulging or ruptured spinal disks, arthritis, or osteoporosis, sciatica, Amyotrophic Lateral Sclerosis (ALS or Lou Gehrig's disease), Seizures or Benign Fasciculation Syndrome; muscle loss, upper motor neuron disease, stroke, MS, arthritis, myositis and polio; and numbness, tingling or painful sensations, peripheral neuropathy caused by autoimmune diseases such as lupus and rheumatoid arthritis, Guillain-Barre syndrome, diabetes, injury, Vitamin B deficiencies and infections such as shingles, Lyme disease, SARS, SARSCov-2, long COVID, loss of smell and taste and HIV.

### BRIEF DESCRIPTION OF THE DRAWINGS

FIG. 1 shows a magnetic stimulation device.

FIG. 2 shows an electric stimulation device.

FIG. 3 shows a battery-operated electric stimulation device.

FIG. 4 shows a graph depicting the colors of noise.

FIG. 5 shows rotating permanent magnetic device

### DETAILED DESCRIPTION OF THE INVENTION

All references to the magnetic stimulation of the present invention equally apply to electric stimulation and vice versa. Magnetic stimulation includes both electromagnetic stimulation and solid (permanent) magnet stimulation.

In one embodiment of the present invention a personalized electromagnetic or electric stimulation protocol is made to a patient's individual neural and muscular measurements.

Chronic neuromuscular pain is often associated with local compensatory muscle spasm. Analgesic treatment should consider muscle relaxation. The present invention involves signal masking at the neuromuscular junction (NMJ) by random electromagnetic stimulation. An NMJ is a chemical synapse between a motor neuron and a muscle fiber. It allows the motor neuron to transmit a signal to the muscle fiber, causing muscle contraction.

Synaptic transmission at the NMJ begins when an action potential reaches the presynaptic terminal of a motor neuron, which activates voltage-gated calcium channels to allow calcium ions to enter the neuron. Calcium ions bind to sensor proteins on synaptic vesicles, triggering vesicle fusion with the cell membrane and subsequent neurotransmitter release from the motor neuron into the synaptic cleft. In vertebrates, motor neurons release acetylcholine (ACh), to bind to nicotinic acetylcholine receptors (nAChRs) on the cell membrane of the muscle fiber.

On the post-synaptic side, the muscle membrane will generate series of electric activities called End Plate Potentials (EPPs). When EPPs reach certain threshold as caused by large amount of ACh from pre-synaptic nerve ending, action potentials on the muscle fiber will occur resulting in contraction. Effective blockage of nerve pulses from reaching to the NMJ, reduction of neural transmitter release, or suppression of EPPs will be able to reduce muscle contraction or relax it from spasm. In the absence of an action potential, ACh vesicles spontaneously leak into the NMJ and cause very small depolarizations in the postsynaptic membrane. This small response is called a miniature end plate potential (MEPP). MEPPs occur spontaneously with random period somewhere about 50 msec. Muscle activities related to MEPPs as measured by electromyography (EMG) vary in frequencies between 20 Hz and 150 Hz (50 msec and 6 msec in period), and peaked at about 70 Hz (14 msec in period).

Random magnetic noise in the EMG frequency band applied to the NMJ should be able to prevent the EPPs from reaching the threshold for muscle contraction and, therefore, relax the spasm and reduce associated pain. An animal study on diaphragm muscle contraction when external stimulation was applied to the connected nerve showed that the force of diaphragm contraction increased with the nerve stimulation frequencies and quickly reduced beyond 40 Hz. High frequency random stimulation is used to relax the muscle spasm to reduce associated pain.

In another embodiment personalized transcutaneous magnetic stimulation, controlled by trains of random TTL pulses limited by individual's EMG frequency bandwidth (or pulse-to-pulse period range), are employed as follows:
(i) One or more channels of digital EMG are recorded and stored for off-line analysis.
(ii) Raw data are bandpass filtered between 20 Hz and 150 Hz to extract the dominant EMG signals at rest.
(iii) Waves at zero-crossings of each complete cycle are depicted to calculate the wave period between each adjacent zero-crossing points.
(iv) The pulse-to-pulse period range acquired from the calculation is then used to generate TTL pulse trains in the white noise pattern within the limit of EMG period variation.

In a further embodiment, the present invention is used to enhance neural synaptic transmission in the central nervous system. In a preferred embodiment repetitive Transcranial Magnetic Stimulation (rTMS) is administered to a patient to treat psychological pathologies or physical conditions described herein. Random magnetic noise in the EEG frequency band of 0.1 to 15 Hz is employed to treat these patients. Preferably, the magnetic stimulation pattern is white noise.

Personalized transcranial magnetic stimulation controlled by trains of random TTL pulses normalized by Gaussian Distribution with an individual's EEG mean period and its standard deviations are employed as follows:
a. One or more channels of digital EEG are recorded and stored for off-line analysis.
b. Raw data are bandpass filtered between 4 Hz and 15 Hz to extract the dominant EEG signals at rest.
c. Waves at zero-crossings of each complete cycle are depicted to calculate the wave period between each adjacent zero-crossing points.
d. Means and standard deviations of the periods of the entire record for each recording channel are calculated.
e. Box-Muller transform based on an individual's mean EEG period and standard deviation are calculated to generate independent, standard normally distributed numbers for each train of TTL sequence.

### Colors of Noise

In addition to the Gaussian noise for the EEG-based stimulation protocol, the following types (colors) of noise, each of which may fit a particular profile of electric activity, such as mEPP or MEG.

Noise is classified by the spectral density, which is proportional to the reciprocal of frequency (f) raised to the power of beta. The power spectral density (watts per Hertz) illustrates how the power (watts) or intensity (watts per square meter) of a signal varies with frequency (Hertz).

$$PSD ∝ \frac{1}{{f}^{β}}$$

White Noise: A defining characteristic of this type of noise is it has a flat power spectral density, meaning it has equal power at any frequency. β=0 for white noise.

Pink Noise: Pink noise is a signal whose power spectral density decreases proportionally to the inverse of the frequency, where the β=1.

Brown Noise: When β=2, the noise is Brownian. Compared with Pink noise, brown noise loses power as frequency increases at a much faster rate than that of pink noise.

Blue Noise: Blue noise is a signal whose power spectral density increases proportionally to the frequency, where the β=−1.

Purple Noise: When β−2, the noise is purple/violet. Purple noise has more energy at higher frequencies.

### Identification of Colors of Noise

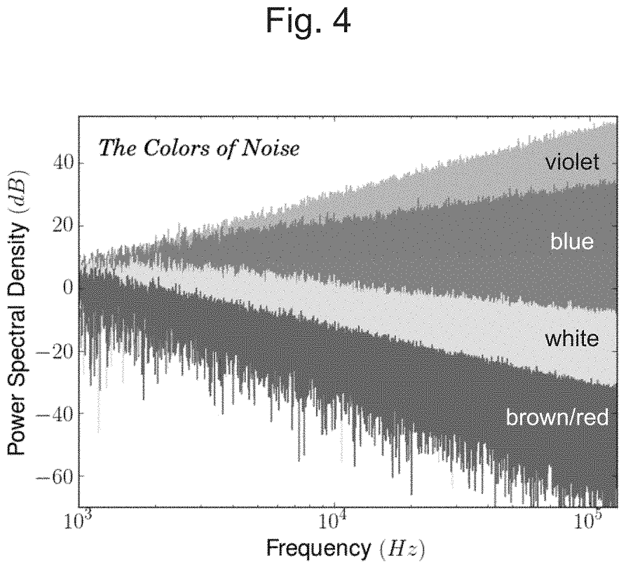

Data taken from a biometric measure→Fourier Transform→Power Spectrum→Curve Fit→Classification of Noise Color
Random sequence generation for the TTL series is a reverse process by weighing the probability of random pulses of frequencies (1/period) according to the color of noise identified from an individual's biometric data. FIG. 4 shows a simulated power spectral densities as a function of frequency for various colors of noise for violet (top), blue, white, pink, brown/red (bottom).

The present examples illustrate the practice of the present invention and should not be construed as limiting its scope.

### Example 1: Treatment of Pain from Shingles

Shingles is a viral infection that causes a painful rash. It most often appears as a single stripe of blisters along a cranial or spinal nerve distribution area. Symptoms often include pain, burning, numbness or tingling, rashes, blisters, and itching. All of these symptoms are associated with the infected nerves. The hibernating herpes viruses tends to stay dormant in the body and reactivate in nerve ganglia. Electromagnetic stimulation treatment with the pulse sequence described in the present patent application over the ipsilateral side of neural ganglia (e.g. spinal nerve ganglia or trigeminal ganglia) of the affected nerves effectively reduces the symptoms. The patient received daily treatment (Mon-Fri) with white noise ipsilateral to the lesion for 2 minutes per treatment and reported that the severity of pain was reduced more than 60% following the first session of treatment and 90% after the 3rd daily treatment. The red blisters of the shingles lesion were reduced more than 50% 10 hours after the first treatment.

### Example 2: Treating a Quadriplegic from a Car Accident

A 24-year-old man suffered a traumatic brain injury from a car accident nine (9) months prior to treatment. His injuries included a hemorrhage in the right hemisphere of his brain that left him a quadriplegic. The patient had uncontrollable rigidity and spasms on his left side—arms and legs. He was Babinski positive. The patient received low frequency random pulses (<5 Hz—white noise) of repetitive transcranial electromagnetic stimulation to the left cortex for 10 seconds every 30 seconds for 10 minutes. Four (4) minutes after this first treatment session, the patient's legs and arms, including hands, became relaxed. This was the first time the patient could move his hands in 9 months from the car accident.

### Example 3: Treating Cerebral Palsy

A four (4) year old boy cerebral palsy patient was unable to move his lower limbs. The patient received low frequency random pulses (<5 Hz—white noise) of electromagnetic stimulation to the bilateral cortex areas 6 seconds every 60 seconds for 30 minutes (5 days per week). After 40 treatments the patient gained 80% muscle tone and could walk with aid of holding onto something, ie, hands of others, a stable holding bar, etc.

### Example 4: Treating Restricted Shoulder Movement

A 40-year-old male patient had restricted shoulder movement in his right shoulder. He could only raise his right arm about 30 degrees. The patient received high frequency random pulses (30-150 Hz—white noise) of electromagnetic stimulation to the affected shoulder blade for alternating 10 second periods for 3 minutes (10 sec stim/10 sec recovery/10 sec stim). Immediately after the 3-minute magnetic stimulation session the patient was able to lift his right arm over his head.

### Example 5: Treating Restricted Neck Movement

A 43-year-old female patient suffered from restricted neck movement. She was only able turn her neck about 20 degrees to each side. The patient received high frequency random pulses (30-150 Hz—white noise) of electromagnetic stimulation to the back of the neck where the restricted area was for alternating 10 second periods for 3 minutes (10 sec stim/10 sec recovery/10 sec stim). Immediately after the 3-minute electromagnetic stimulation session, the patient regained full range of motion in her neck.

### Example 6: Treating a Stroke Patient

A 52-year-old male stroke patient had a stroke one year prior to the present electromagnetic stimulation treatment. Since the stroke the patient was unable to move his left arm and left leg. The patient received low frequency random pulses (<5 Hz—white noise) of electromagnetic stimulation to the right motor cortex of the brain for 6 seconds every 60 seconds for 30 minutes. After this initial treatment the patient was able to move his left leg and left arm.

### Example 7: Treating a Scuba Diving Accident (Bends)

A 41-year-old male experienced deep sea scuba diver involved in a dive greater than 150 feet below the surface of the ocean resurfaced too quickly that resulted in paralysis from his waist down for a period of 7.5 years. The patient received low frequency random pulses (<5 Hz—white noise) of repetitive transcranial electromagnetic stimulation to the bilateral cortex area for 6 seconds every 60 seconds for 30 minutes (5 days per week). Within 2 weeks after the start of treatment the diver was able to feel his legs and move his toes. At the end of five (5) weeks the diver was able to stand up.

Another aspect of the present invention is the apparatus or hardware used to deliver electromagnetic stimulation to the patient. This includes magnetic stimulation devices and electrostimulation devices.

A magnetic stimulation apparatus includes a magnetic field generator, a power source configured to energize the magnetic field generator to produce magnetic field pulses and a digital program that directs the magnetic field generator to produce variable pulses according to the present invention as described herein. The variable pulses can be based on the mean and standard deviations or a selected frequency bandwidth of one or more biometric variables normalized by following idealized noise patterns or a Gaussian distribution.

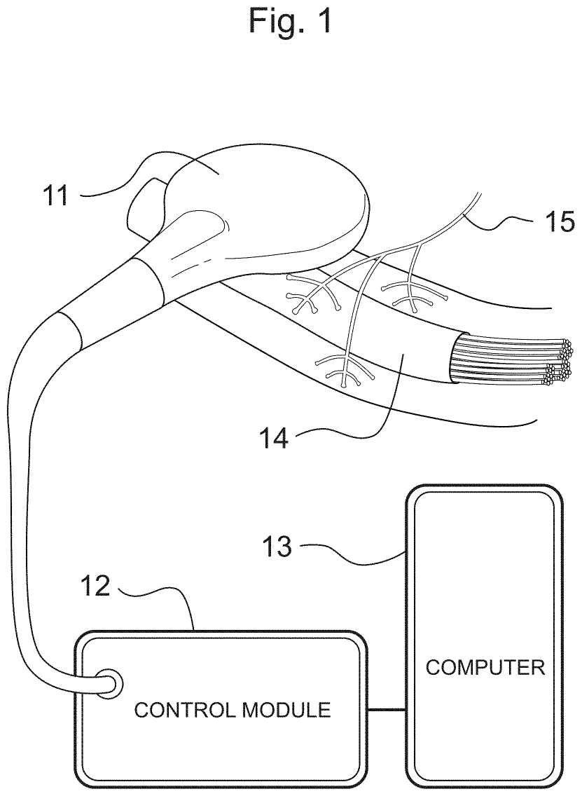

FIG. 1 shows a magnetic stimulation device of the present invention that contains a coil 11 and a control module 12 that includes a power source (not shown). Typically, the control module that controls the TTL pulses and wires that extend up to the coil 11. When the power source (not shown) is turned on, an electric current pass through the coiled wires producing a magnetic field. A software or hardware program 13 directs the apparatus to produce variable pulses according to the present invention including the low frequency and high frequency ranges disclosed herein. In administering the magnetic stimulation treatment to a patient, the encased magnet coil component is positioned on or in close proximity to the body part receiving the treatment. Muscle fibers 14 and neurons 15 are shown to illustrate the muscle-synaptic transmission between neuron and muscle fibers in peripheral tissue.

An electric stimulation apparatus includes two or more electrode pads that adhere to the skin, a power source configured to produce an electric current in said electrodes and a digital program that directs the electric stimulation of the variable pulses according to the present invention as described herein. The variable pulses can be based on the mean and standard deviations of one or more biometric variables normalized by following idealized noise patterns or a Gaussian distribution. In a preferred embodiment of an electric stimulation apparatus, the power source of the electric stimulation apparatus is a battery which provides ease of use in medical treatments including an over-the-counter at-home transcutaneous electric nerve stimulation (TENS unit) device. In administering the electric stimulation treatment to a patient, the electrode pads are positioned contiguous or adjacent to the painful body part receiving the treatment.

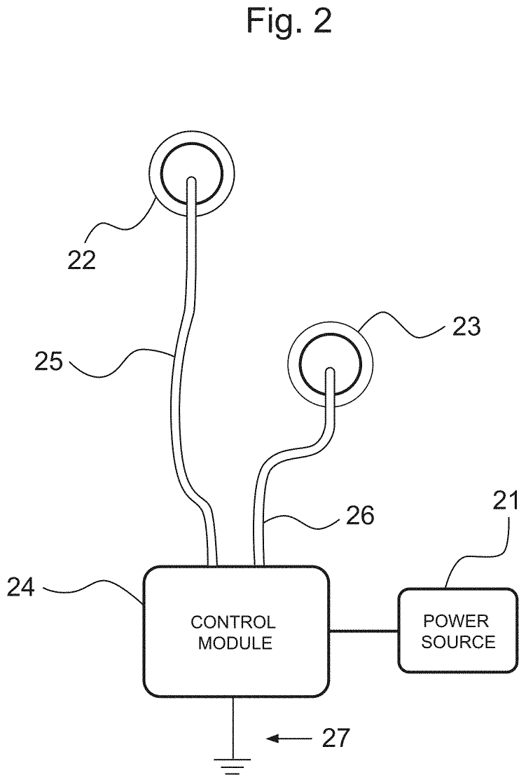

FIG. 2 shows an electric stimulation device 20 of the present invention that contains an electric power source 21, a pair of electrode pads 22 and 23 connected to a control module 24 with wires 25 and 26. The control module is grounded 27. A digital or computer program in the control module 24 directs the electric stimulation of the variable pulses according to the present invention including the low frequency and high frequency ranges disclosed herein. In administering the electric stimulation treatment to a patient, the electrodes are positioned contiguous or adjacent to the painful body part receiving the treatment.

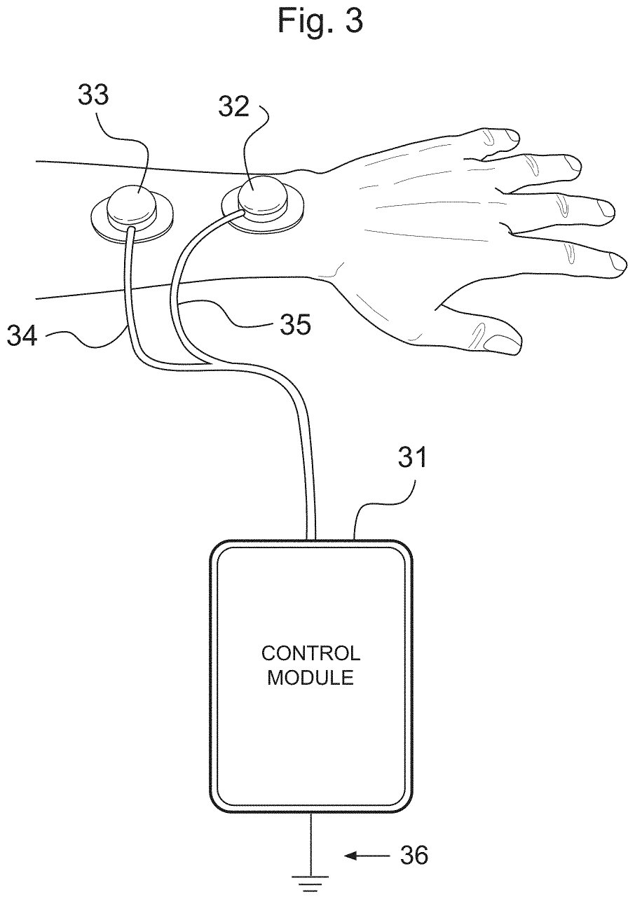

FIG. 3 shows a battery powered electric stimulation device of the present invention that contains an electric pulse generator 31 powered by a battery, a pair of electrode pads 32 and 33 connected to the control module 31 with wires 32 and 33. The control module is grounded by 36. A digital or computer program in the control module directs the electric stimulation of the variable pulses according to the present invention including the low frequency and high frequency ranges disclosed herein. In administering the electric stimulation treatment to a patient, the electrodes are positioned contiguous or adjacent to the painful body part receiving the treatment.

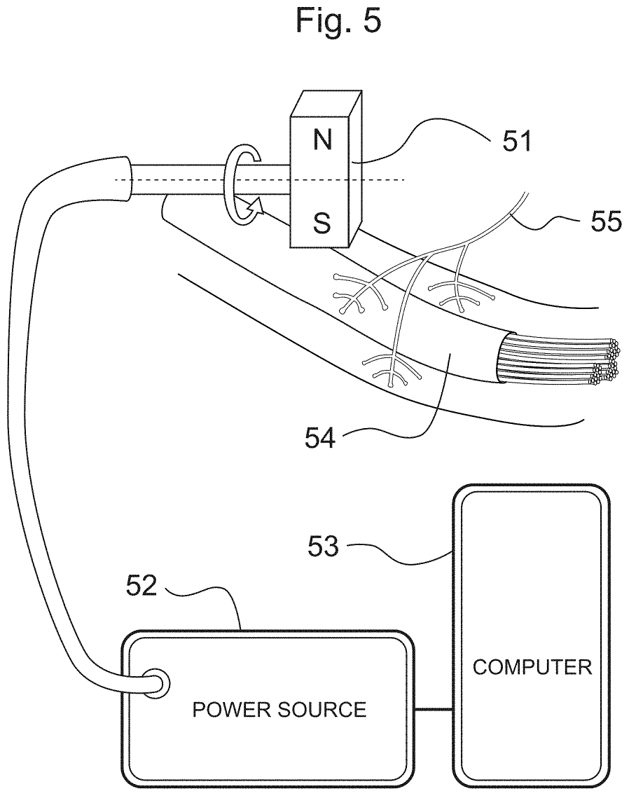

FIG. 5 shows a permanent magnetic stimulation device of the present invention that contains a permanent magnetic 51 and a power source 52 When the power source is turned on, the permanent magnet rotates to produce a magnetic field. A software or hardware program 53 directs the apparatus to produce variable rotation speeds according to the present invention including the low frequency and high frequency ranges disclosed herein. In administering the magnetic stimulation treatment to a patient, the encased (not shown) permanent magnet component is positioned on or in close proximity to the body part receiving the treatment. Muscle fibers 54 and neurons 55 are shown to illustrate the muscle-synaptic transmission between neuron and muscle fibers in peripheral tissue.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of masking a signal at a peripheral neuromuscular junction (NMJ) in a patient which comprises applying magnetic stimulation to the patient wherein the magnetic stimulation comprises variable magnetic pulses.

2. The method claim 1 wherein the variable magnetic pulses are random.

3. The method of claim 1 wherein the magnetic pulses are delivered in a designed pattern of noise.

4. The method of claim 3 wherein the pattern of noise is white noise, pink noise, violet noise, blue noise, brown noise or red noise.

5. The method of claim 4 wherein the variable magnetic pulses are a heterogenous mixture of magnetic pulses over a frequency range of from 30 to 150 Hz and a wide pulse interval of from 33.3 msec to 6.7 msec.

6. The method of claim 5 wherein the stimulation masks neuronal transmission.

7. The method of claim 6 wherein the magnetic stimulation is used to treat pain.

8. A method of enhancing a neuronal-synaptic signal in a patient which comprises applying variable repetitive transcranial magnetic stimulation (rTMS) to the patient.

9. The method claim 8 wherein the variable magnetic pulses are random.

10. The method of claim 8 wherein the magnetic pulses are delivered in a pattern of noise.

11. The method of claim 10 wherein the pattern of noise is white noise, pink noise, violet noise, blue noise, brown noise or red noise.

12. The method of claim 11 wherein the variable magnetic pulses are a heterogenous mixture of magnetic pulses over a frequency range of from 0.1 to 15 Hz and a wide pulse interval of from 10,000 msec to 66.7 msec.

13. The method of claim 12 wherein the stimulation enhances transmission of signals between neurons.

14. The method of claim 13 wherein the magnetic stimulation is used to treat psychological disorders or physical conditions.

15. A method of magnetic neuromodulation in a patient which comprises:
a. measuring a biometric character of a patient resulting in a biometric data set,
b. comparing the biometric data set of (a) with a normative database to determine if the patient needs neuromodulation,
c. analyzing the biometric data set to identify characteristics of distribution probabilities of one or more variables resulting in mean and standard deviation of pulse period values,
d. administering magnetic stimulation to the patient wherein the magnetic stimulation comprises variable pulses based on one or more mean and standard deviations of the biological variables selected from the group consisting of an EEG, an EMG or a spinal cord electric pulse frequency measurement.

16. The method of claim 13 wherein the biometric character is an EEG.

17. The method of claim 13 wherein the biometric character is an EMG.

18. A method of relieving pain in a patient which comprises applying electromagnetic stimulation to the patient wherein the magnetic stimulation comprises variable electromagnetic pulses.

19. The method of claim 16 wherein the magnetic pulses in a preferred pattern of noise.

20. The method of claim 17 wherein the pattern of noise is white noise.

21. The method of claim 18 wherein the white noise is a heterogenous mixture of magnetic pulses over a selected frequency range of from 30 to 150 Hz and a pulse interval of from 33.3 msec to 6.7 msec.

22. A magnetic stimulation apparatus which comprises:
a. a magnetic field generator,
b. a power source configured to energize the magnetic field generator to produce magnetic field pulses and
c. a digital program that directs the magnetic field generator to produce variable pulses based on the mean and standard deviations of one or more variables normalized by following idealized noise patterns.

23. An electric stimulation apparatus which comprises:
a. two or more electrode pads,
b. a power source configured to produce an electric current in said electrode pads and
c. a digital program that directs the electric stimulation of variable pulses based on the mean and standard deviations of one or more variables normalized by following idealized noise patterns.

24. The electric stimulation apparatus of claim 23 where the power source is a battery.

25. The apparatus of claim 23 which is a transcutaneous electrical nerve stimulation device (TENS Unit).

26. A method of treating pain in a patient which comprises applying transcutaneous magnetic stimulation proximate to the area of the pain wherein the magnetic stimulation is a heterogenous random mixture of magnetic pulses over a wide frequency range of from 30 to 150 Hz and a wide pulse interval of from 33.3 msec to 6.7 msec wherein each frequency and pulse interval have an equal opportunity to be selected in each pulse train

27. The method of claim 26 the magnetic stimulation has a white noise pattern.

28. The method of claim 26 wherein the patient's pain is caused by shingles.

29. A method of magnetic neuromodulation in a patient which comprises:
e. measuring a biometric character of a patient resulting in a biometric data set,
f. comparing the biometric data set of (a) with a normative database to determine if the patient needs neuromodulation,
g. analyzing the biometric data set to identify characteristics of distribution probabilities of one or more variables resulting in mean and standard deviation of pulse period values,
h. administering magnetic stimulation to the patient wherein the magnetic stimulation comprises variable pulses based on one or more mean and standard deviations of the biological variable.

30. The method of claim 29 wherein the standard deviation is from 0.1 to 10.0 standard deviations.

31. The method of claim 30 wherein the variables comprise a pulse period variable or a biometric of heart rate variability, EEG variability, EMG variably or spinal cord measurement variability.

32. The method of claim 29 wherein the neuromodulation inhibits NMJ transmission between neurons and muscle fibers.

33. The method of claim 29 wherein the neuromodulation enhances synaptic transmission between neurons.

34. A method of neuromodulation in a patient which comprises applying electric stimulation to the patient wherein the electric stimulation comprises variable electric pulses.

35. The method claim 34 wherein the variable electric pulses are random.

36. The method of claim 34 wherein the electric pulses mimic a pattern of noise.

37. The method of claim 36 wherein the pattern of noise is white noise, pink noise, violet noise, blue noise, brown noise or red noise.

38. The method of claim 35 wherein the variable electric pulses are a heterogenous mixture of electric pulses over a random frequency range of from 30 to 150 Hz and a wide pulse interval of from 33.3 msec to 6.7 msec.

39. A method of neuromodulation in a patient which comprises applying electric stimulation to the patient wherein the electric stimulation has a zero-mean value, a constant variance and is uncorrelated in time.

40. The method of claim 39 wherein the neuromodulation is used to treat pain.

41. The method of claim 40 wherein the magnetic stimulation is a heterogenous mixture of electric pulses over a random frequency range of from 30 to 150 Hz and a wide pulse interval of from 33.3 msec to 6.7 msec.

42. The method of claim 41 wherein the frequency range is 50 to 100 Hz and the pulse interval is 20 msec to 10 msec.

43. The method of claim 6 wherein the magnetic stimulation is used to treat shingles.
